(19) United States
(12) Patent Application Publication
(10) Pub. No.: US 2009/0123959 A1
(43) Pub. Date: May 14, 2009

(54) MICROORGANISM DISCRIMINATOR AND METHOD

(75) Inventors: Stephen Joseph VESPER, Cincinnati, OH (US); Alexander Nicholas VESPER, Cincinnati, OH (US)

Correspondence Address: STEIN, MCEWEN & BUI, LLP, 1400 EYE STREET, NW, SUITE 300, WASHINGTON, DC, 20005, US

(73) Assignee: U.S. Environmental Protection Agency, Washington, DC (US)

(21) Appl. No.: 12/172,519

(22) Filed: Jul. 14, 2008

Related U.S. Application Data
(60) Provisional application No. 60/996,043, filed on Oct. 25, 2007.

(51) Int. Cl. C12Q 1/02; C12M 1/00

(52) U.S. Cl. 435/29; 435/288.7

(57) ABSTRACT

A microorganism discriminator is disclosed, including a housing to incubate a sample in low-light conditions; a illuminator to irradiate the sample with a monochromatic blue light; an injector disposed in the housing, to deliver a viability discriminating dye to the sample; and a base connected to the housing and the illuminator, to transport the sample to the housing and to the illuminator. A method of discriminating viable microorganisms in a sample is disclosed, the method including: applying a sample to a filter; applying a viability discriminating dye to the filter, in a low-light environment; incubating the sample in the low-light environment; illuminating the filter with monochromatic blue light; and performing quantitative polymerase chain reaction (QPCR) on the sample.

### CROSS-REFERENCE TO RELATED APPLICATION

This application claims the benefit of U.S. Provisional Patent Application No. 60/996,043, entitled: Microorganism Discriminator, filed on Oct. 25, 2007, the disclosure of which is incorporated herein, by reference.

### GOVERNMENT INTEREST

This invention was made with Government support from U.S. Environmental Protection Agency (EPA), through its Office of Research and Development. The Government has certain rights in this invention.

### BACKGROUND OF THE INVENTION

1. Field of the Invention

The present teachings relate to an apparatus to detect microorganisms, and a method of detecting microorganisms.

2. Description of the Related Art

Microorganisms can propagate in nearly any environment, and many microorganisms are pathogenic to humans. For example, fungal infections can have fatality rates as high as 50-90%. Microorganism detection can be critical in hospitals, where immune-compromised patients can be especially susceptible to colonization. Therefore, the detection of pathogenic microorganisms is critical.

Historically, culture-based detection methods have been used to detect viable microorganisms. However, depending on the microorganism, it can take days to weeks to quantify microorganisms by culturing. For example, culture-based methods of fungal analysis can take days to weeks. If viable microorganisms are detected, steps to reduce or eliminate these organisms may be possible, before exposures can occur.

However, culture-based methods can only detect live microorganisms. In some settings where anti-microbial treatments are proactively undertaken, such as in a hospital, it may be critical to detect not only whether an infectious microorganism, such as Aspergillus fumigatus, is present in a sample, but also whether the cells are viable, and therefore, potentially infectious, after a treatment has been applied. Similarly, Legionella pneumophila in cooling tower water can cause fatal cases of pneumonia, and it may be critical to know whether cooling tower water treatments are successful in controlling such a microorganism.

A number of “viability” stains or dyes have been used to identify viable microbial cells (Arzumanyan and Ozhovan 2002; Oh and Matsuoka, 2002; Jin et al., 2005). Some of these stains or dyes penetrate the porous membranes of dead cells, but are unable to penetrate the intact membranes of live cells. Solid phase cytometry has been used to measure viable fungi in water samples, but the species of fungi can not be determined by this method (De Voss and Nelis, 2006).

Another method of detecting microorganisms involves the use of quantitative polymerase chain reaction (QPCR). QPCR is a more rapid and sensitive method for testing environmental samples than culture-based techniques. However, QPCR does not differentiate between viable and non-viable cells. With the increased use of species specific QPCR assays, attempts have been made to link viability tests with the QPCR process.

Propidium monoazide (PMA) has been successfully used to differentiate viable and non-viable bacteria, in conjunction with QPCR (Nocker et al., 2006). PMA is able to enter the membranes of heat-killed bacterial cells, and intercalate the DNA therein, or bind to any free DNA in a sample. PMA inhibits the activity of Taq polymerase, during QPCR analysis.

A number of viability stains and associated instruments have been created, but have various drawbacks, and are not compatible with QPCR analysis. For example, solid phase cytometry has been used, but this technique does not identify the species of microorganism (see De Vos and Nelis, J. Microbiological Methods 2006; 67:557-565.)

Recently, propidium monoazide (PMA) has been used to distinguish live and dead bacterial cells (Nocker A, Sossa K E, Camper A K. Molecular monitoring of disinfection efficacy using propidium monoazide in combination with quantitative PCR. J. Microbiol. Methods. 2007 August; 70(2):252-60. Epub 2007 May 1) The taught process is fully manual, and has many limitations that prevent automation.

Therefore, there is a need to determine the type and number of cells of an organism that are present in a sample, and also whether the cells are alive. There is also a need for an apparatus that can automatically and rapidly perform such a determination.

### SUMMARY OF THE INVENTION

According to various embodiments, the present teachings relate to a microorganism discriminator comprising: a housing to incubate a sample in low-light conditions; an illuminator to irradiate the sample with a monochromatic blue light; an injector disposed in the housing, to deliver a viability discriminating dye to the sample; and a base connected to the housing and the illuminator, to transport the sample to the housing and to the illuminator.

According to various embodiments, the present teachings relate to a method of discriminating viable microorganisms in a sample, the method comprising: applying a sample to a filter; applying a viability discriminating dye to the filter, in a low-light environment; incubating the sample in the low-light environment; irradiating the filter with monochromatic blue light; and performing quantitative polymerase chain reaction (QPCR) on the sample.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a front perspective view of a microorganism discriminator;

FIG. 2 is a rear perspective view of the microorganism discriminator;

FIG. 3 is a perspective view of a frame of the microorganism discriminator;

FIG. 4 is a perspective view of an illuminator of the microorganism discriminator;

FIG. 5 is a perspective view of a sample plate; and

FIG. 6 is a perspective view of an injector array.

### DETAILED DESCRIPTION OF THE EXEMPLARY EMBODIMENTS

Reference will now be made in detail to the exemplary embodiments of the present invention, examples of which are illustrated in the accompanying drawings, wherein like reference numerals refer to the like elements throughout. The exemplary embodiments are described below, in order to explain the aspects of the present invention, by referring to the figures.

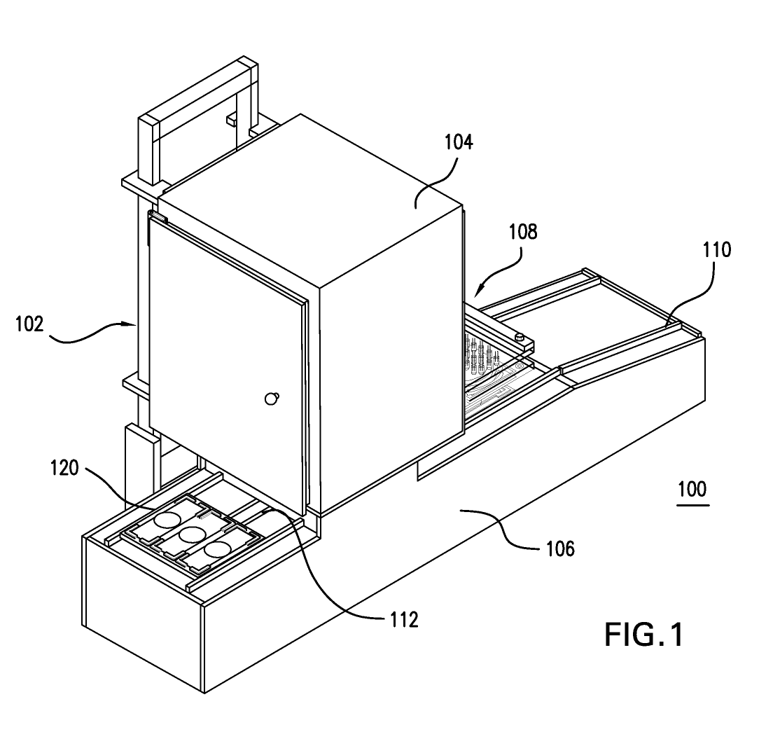

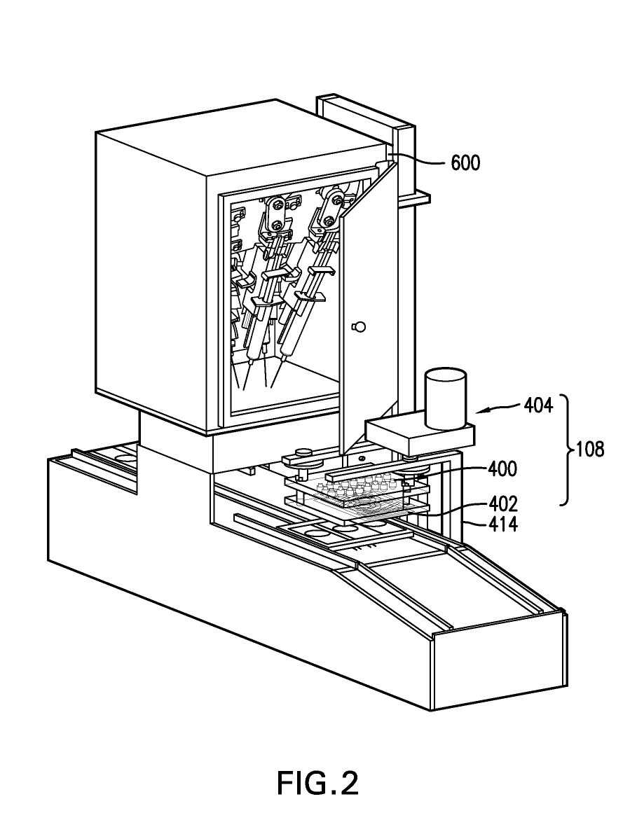

FIGS. 1 and 2 are front and rear perspective views of a microorganism discriminator 100, according to an exemplary embodiment of the present invention. The discriminator 100 includes: a frame 102; a housing 104 mounted to the frame 102; a base 106 connected to the frame 102, and an illuminator 108 connected to the base 106.

A track 110 and a conveyor 112 are disposed on the base 106. A sample plate 120 can be conveyed along the track 110, by the conveyor 112. The conveyor 112 can be a motorized belt, for example, or can be any suitable device that can move a sample plate 120, relative to the base 106. The illuminator 108 is disposed above the track 110, adjacent to the housing 104. While depicted as having a stationary frame 102 and illuminator 108, it is understood that one or both of the frame 102 and the illuminator 108 can move relative to the base 106, in addition to, or instead of the conveyor 112.

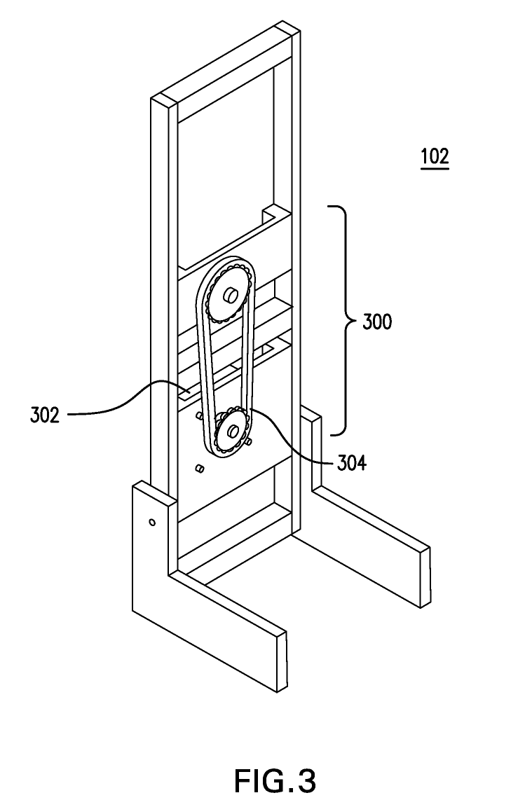

As shown in FIG. 3, the frame 102 includes an elevator 300 to raise and lower the housing 104. The elevator 300 can be any device capable of controlling the vertical orientation of the housing 104. For example, the elevator can include a motor 302 and a chain 304 that is driven by the motor 302, as shown. The chain 304 can be connected to the housing 104, such that when the motor 302 is driven, the housing 104 is moved between a lowered position, as shown in FIG. 1, and a raised position, as shown in FIG. 2. However, the invention is not limited thereto.

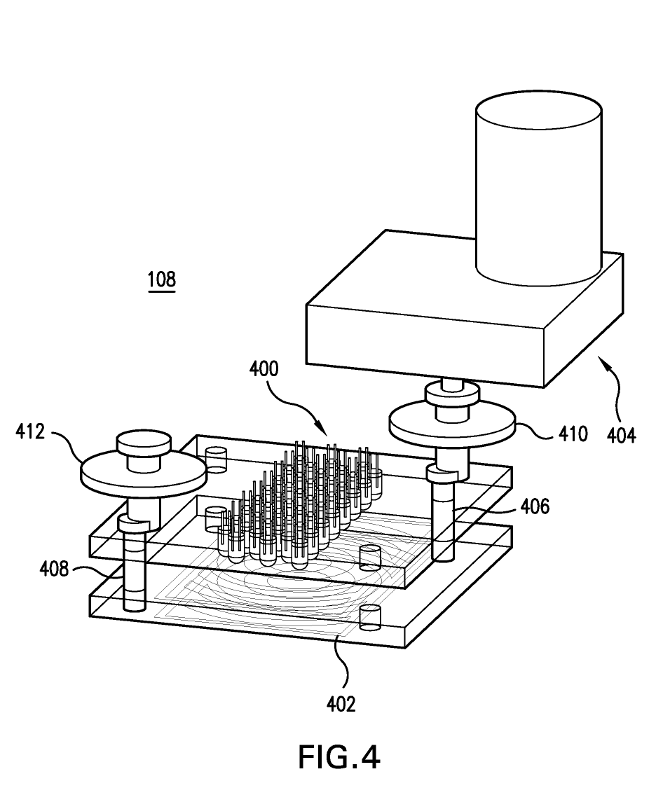

As shown in FIG. 4, the illuminator 108 includes a light source 400, a lens 402, and a driver 404. The light source 400 can radiate a monochromatic blue light to the lens 402. The blue light can have a wavelength ranging from about 445 to about 485 nm. According to some embodiments, the blue light can have a wavelength of 470 nm.

The light source 400 can be any light source that can produce a monochromatic blue light. For example, the light source 400 can be a bundle of light emitting diodes (LEDs), or one or more blue lasers. The light source 400 can be connected to a power source (not shown). The lens 402 collects and focuses the blue light onto the sample plate 120, when the sample plate 120 is positioned below the illuminator 108, by the conveyor 112. The lens 402 can be a Fresnel lens, for example. While depicted as a single lens, it is understood that the lens 402 can be multiple lenses that form an optical focusing system.

The light source 400 and the lens 402 can be connected by first pins 406, 408. The shown pins 406, 408 can be connected to pivot rods 410, 412, which are rotatably disposed on a bracket 414 mounted to the base 106, as shown in FIG. 2. The pivot rod 410 can be rotated by the driver 404, such that the light source 400 and the lens 402 oscillate. The oscillation can insure that a sample disposed there below, is evenly illuminated.

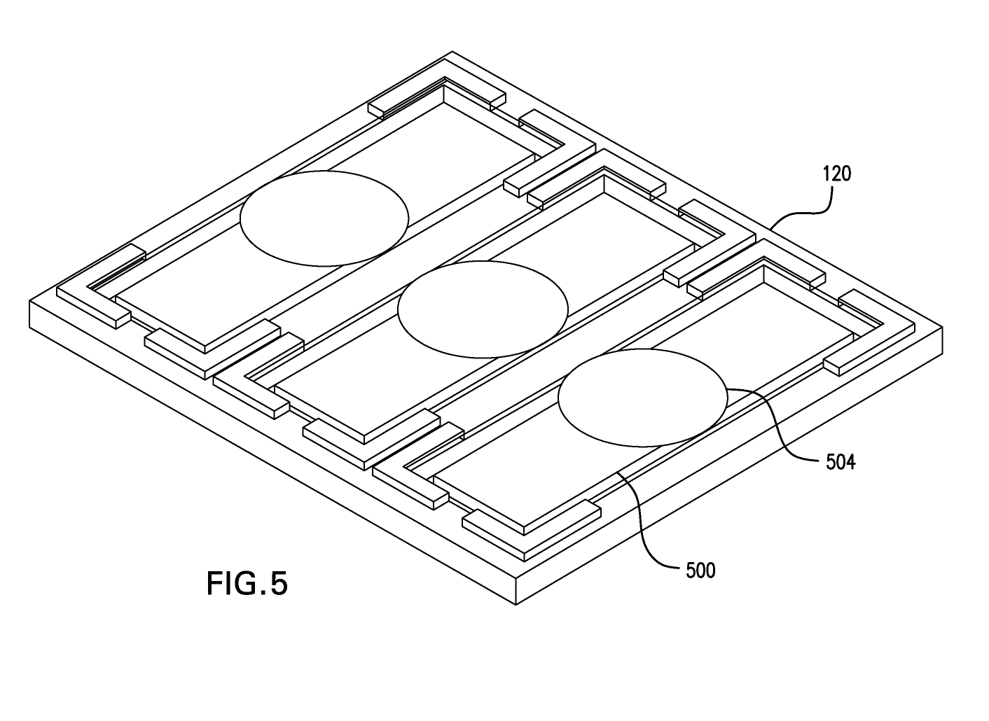

As shown in FIG. 5, the sample plate 120 can be configured to hold one or more slides 500. The shown slides 500 can be welled slides. Filters 504 can be disposed on the wells of the slides 500. The filters 504 can be any filter that is compatible with a quantitative polymerase chain reaction (QPCR) assay. For example, the filters 504 can be polycarbonate filters, or Teflon® filters. The wells can be filled with a buffer, for example, phosphate buffered saline (PBS).

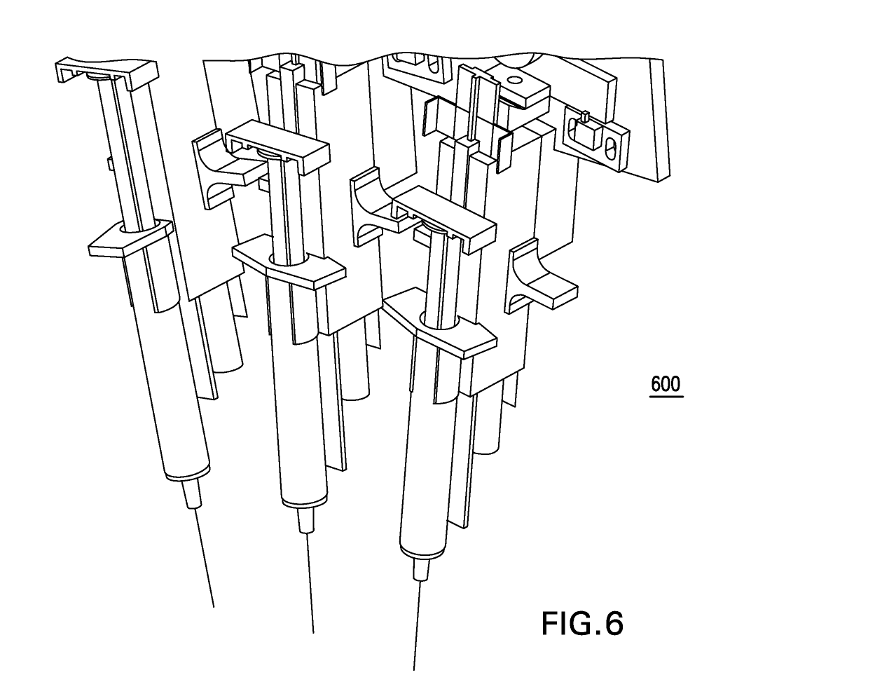

As shown in FIGS. 2 and 6, the discriminator 100 can include an injector 600, which is disposed in the housing 104. The injector 600 can be a liquid injector that is suitable for accurately dispensing small amounts of a liquid. For example, the injector 600 can be a number of pipettors or syringes. For example, as shown in FIG. 6, the injector 600 can include one or more arrays of pipettors. The injector 600 can inject a viability discriminating dye onto the filters 504.

As referred to herein, a viability discriminating dye is a DNA intercalating dye that readily penetrates dead or membrane compromised cells, but does not penetrate live cells that have intact cell membranes. Once the dye intercalates DNA of a sample, the dye can be crosslinked to the DNA, by exposure to light. The cross-linked dye prevents the associated DNA from being amplified, during a polymerase chain reaction (PCR) procedure. The dye can inhibit the activity of a DNA polymerase during PCR.

According to an exemplary embodiment, the dye can be Propidium monoazide (PMA). However, other dyes that satisfy the above conditions can also be used. The PMA can be cross-linked to DNA using white light, as disclosed in Nocker et al., Journal of microbiological Methods, 67 (2006) 310-320, the disclosure of which is incorporated herein, by reference. However, the use of white light results in the excessive production of heat, which can damage a sample. Therefore, the PMA can be cross-linked using the blue light produced by the illuminator 108. The blue light produces optimal cross-linking, with minimal heat production.

Referring again to FIGS. 1 and 2, the present teachings encompass a method of detecting viable cells. The method comprises, isolating a sample of microorganisms on a filter 504. For example, the filter 504 can be placed in a holder, and then water or air containing the sample can be vacuumed through the filter 504. The filter 504 can then be removed from the holder, and placed it on the slide 500. The holder can be a button sampler, or the like, for example.

The sample may be a sample that has been previously undergone a biocidal treatment. For example, the sample may have been heat treated, or an antibiotic/antifungal agent may have been applied to the sample. The microorganisms of the sample can be single-cell organisms, or multi-cellular organisms. For example, the microorganism can be bacteria, fungi, protozoa, viruses, or the like. While the present teachings are generally applicable to samples of cellular microorganisms, the present teachings can also be applied to non-cellular organisms, such as viruses. Viruses have protein coats, or capsules, rather than membranes, which may make a discriminating dye like PMA applicable, if the protein coats or capsules can are, or can be made to be, selectively porous to PMA, or another discriminating dye.

The filter 504 is positioned on the well of the slide 500. The well can be filled with a buffer, such as phosphate buffered saline (PBS). A number of similarly prepared slides 500 can be positioned on the sample plate 120. The sample plate 120 can be positioned on the base 106, in the track 110.

The sample plate 120 is conveyed, by the conveyor 112, to a position under the housing 104. The elevator 300 then lowers the housing 104 over the sample plate 120. When lowered, the housing 104 prevents light from reaching the sample plate, (i.e., keeps the samples on the sample plate 120 in a low-light condition).

The injector 600 injects the discriminating dye onto the filters 504 of the sample plate 120. The PBS buffer in the wells of the slides 500 prevents the filters 504 from drying out, and facilitates the application of the discriminating dye to the filters 504. In this way, the discriminating dye is evenly applied to the filters 504.

The filters 504 are then incubated, to allow the discriminating dye to intercalate DNA in the samples. The filters 504 can be incubated for from about 10 to about 30 minutes. The housing 104 can be humidified during the incubation. The housing 104 may also control the temperature at which the samples are incubated. However, the humidity and/or temperature control may not be performed in all aspects.

Once the samples are incubated, the housing 104 is then raised, and the sample plate 120 is conveyed under the illuminator 108. The blue light produced by the light source 400, is focused by the lens 402, and radiated to the filters 504, for between about 5 and about 15 minutes. During the radiation, the driver 404 can be used to optionally oscillate the light source 400 and the lens 402. The oscillation can be used to insure that all portions of the filters 504 are sufficiently illuminated. The blue light cross-links the discriminating dye to DNA present in the samples, and inactivates any residual discriminating dye.

The sample plate 120 is then further conveyed along the track 110. The filters 504 are removed from the slides 500, for example, by aseptically folding the filters 504. The filters 504 are then inserted into sample tubes for quantitative PCR (QPCR) analysis. Methods have been reported previously for performing QPCR analyses (Roe et al., 2001; Haugland et al., 2002; Brinkman et al., 2003; Haugland et al., 2004), the disclosures thereof, are herein incorporated by reference.

While not required, a controller (not shown) can coordinate the actions of the conveyer 112, the injector 600, the elevator 300, and/or the illuminator 108, so as to automate the process. As such, aspects can be embodied as a mechanical controller, or through software or firmware, using one or more processors.

### EXPERIMENTAL EXAMPLES

### Example 1

Fungal cultures (condia) were grown on potato dextrose agar (PDA), at 23° C., until the cultures sporulated. The conidia were harvested, by adding approximately 5 ml of a sterile 0.5% Tween 80 solution, and gently rubbing the surface of the culture dish with a sterile cotton swab. The suspension of spores was recovered, and filtered through sterile Whatman 541 filter paper, held in a Buchner funnel. During constant mixing on a stir plate, the cell suspension was aliquoted into sterile 0.6 ml microfuge tubes, and frozen at −80° C., until used.

The quantification of the culturable cells was determined, by plating the conidia suspensions on PDA, and incubating plates at 23° C., until the colonies could be counted. The “culturable” population for each species of fungus was based-on the average number of colonies formed (CFUs) on replicate PDA plates.

Propidium monoazide (phenanthridium, 3-amino-8-azido-5-[3-(diethylmethylammonio)propyl]-6-phenyl dichloride; Biotium, Inc., Hayward Calif.) was resuspended in 1 mg per 65.4 μl of dimethyl sulfoxide (DMSO) (SIGMA-ADRICH, St. Louis, Mo.) and distributed into 5 μl aliquots into brown microfuge tubes, then held, at −20° C., until needed. To produce a 30 mM working solution, 600 μl of sterile PBS was added to one of these microfuge tubes.

Assay of Simulated Water and Air Samples

For each test of a particular fungus, a conidial suspension tube (described above) was recovered from the freezer, and 10 μL resuspended in 1 ml of PBS in a sterile 2 ml “Safe-lock” tube (22-60-004-4; PGC Scientific, Fredrick, Md.). The suspension was thoroughly mixed, and 0.5 ml of the suspension was recovered, and placed in an identical tube. The tubes were labeled “Dead” and “Live”. The tube labeled “Dead” was placed in a heat-block (Multi-Blok®, Lab Line, Melrose, PK, IL) at 85° C., for 1 hr. The “Live” labeled tube was held in the refrigerator.

In the test of the mixed species suspensions of cells, 10 μL from tubes of each of the six fungal species was resuspended in 940 μL of PBS (for a total of 1 ml), in a sterile 2 ml tube. The suspension was mixed and split, as described above, into “Dead” and “Live” and the heat treatment described above used.

Water and air samples were collected for QPCR analysis, using polycarbonate filters (Brinkman et al., 2003; Neely et al., 2004; Meklin et al., 2007; Vesper et al., 2007). To simulate these kinds of samples, polycarbonate filters were spiked with the “Dead” and “Live” conidial suspensions for testing. To the middle well of a three well (14 mm diameter well), heavy Teflon® coated slide (10-12; Celine, Erie Scientific Co., Portsmouth, N.H.) was added 30 μL of PBS. A 25 mm polycarbonate filter having a 0.8 μm pore size (Osmonics Inc., Minnetonka, Minn., USA) was asceptically placed directly on the well containing PBS, and the process repeated for each treatment of “Live-PBS”; “Live-PMA”; “Dead-PBS”; and “Dead-PMA”.

Using the “Dead” and “Live” suspensions (prepared and treated as described above), 10 μL of the suspension was added to the filter on the glass slide. Then in very low-light, 10 μL of either PBS or PMA was added to the filter on the slide. The slide was transferred into a light tight black-box (humidified with containers of warm water), and incubated for 20 min.

After incubation, the filters were exposed to two bundles of eight blue light-emitting diodes (LED) (276-316; 5 mm, 3.7 v, 20 mA, 2600 mcd, Radio Shack, Fort Worth, Tex.), for 10 min. The light from the LEDs was focused onto the filter, using a Fresnel lens (Magnavision, FGX International, Smithfield, R.I.).

After the light exposure, the filters were asceptically recovered, by folding the filters, and inserting the same into 2 ml screw cap tubes (PGC #506-636), hereafter called the “bead-beating tube,” containing 0.3 g+/−0.01 of glass beads (SIGMA# G-1277). Then 200 μL of lysis buffer from the GeneRite DNA-EZ® kit (KC101-04C-50; Gene-Rite, Kendal Park, N.J.) was added to each tube containing a filter. Each well (where the filter had been) was washed five times, each wash consisting of 40 μL of lysis buffer, for a total of 400 μL lysis buffer in each bead-beating tube.

The DNA from the fungal cells was extracted as follows. The bead-beating tube was placed in a “Mini-bead Beater” (Biospec Products, Bartlesville, Okla.), and shaken at maximum speed for 1 min. The bead-beating tube was then centrifuged for 1 min, at 12,000 rpm, in a microcentrifuge. The liquid recovered above the beads (approximately 240 μL) was placed in the DNA-EZ® kit “pre-filter,” and centrifuged for 1 min, at 7,000 rpms. The filtrate was recovered, and 600 μL of DNA-EZ Binding Buffer® was added to the filtrate. This mixture was then added to the DNAsure® column from the kit, inserted into a new collection tube, and centrifuged for 1 min, at 12,000 rpm. The column was washed twice with 500 μL of EZ-Wash Buffer® from the kit. The DNA was recovered from the DNAsure® column, by adding 100 μL of the DNA Elution Buffer® from the kit, in two consecutive steps, with centrifuging for 1 min, at 12,000 rpm, for a final volume of 200 μL of purified DNA solution.

Quantitative PCR (QPCR) Analysis of Samples

Methods have been reported previously for performing QPCR analyses (Roe et al., 2001; Haugland et al., 2002; Brinkman et al., 2003; Haugland et al., 2004) which are incorporated herein by reference. Each treatment test was repeated three times, with replicate analyses of each extract. 95% confidence intervals were calculated for each of the treatment comparisons.

The Q PCR analysis was performed on the Roche 480 Light Cycler® using the Roche ERMI Kit® reagents (Roche Diagnostics Co, Indianapolis, Ind.). All primer and probe sequences, as well as known species comprising the assay groups, are described in the document entitled, EPA Technology for Mold Identification and Enumeration, last updated Oct. 30, 2007.

### Results

TABLE 1

| Disease | Culture | “Live” | “Dead” |
|---|---|---|---|
| Fungal Species | Collection and # | CFU/10 μl | CFU/10 μl |
| Aspergillosis | | | |
| Aspergillus terreus | ATCC 1012 | 3.3 × 10⁵ | 1.1 × 10² |
| A. fumigatus | NRRL 163 | 4.6 × 10⁵ | 1.2 × 10³ |
| A. flavus | ATCC 16883 | 1.3 × 10⁵ | 4.0 × 10² |
| Mucormycosis (Zygomycosis) | | | |
| Mucor racemous | NRRL 1428 | 2.3 × 10⁴ | 3.5 × 10¹ |
| Rhizopus stolonifer | ATCC 14037 | 8.4 × 10⁴ | 0 |
| Hyalohyphomycosis | | | |
| Paecilomyces variotti | ATCC 22319 | 5.9 × 10⁴ | 1.2 × 10² |

Table 1 shows fungal culture and source and concentration of conidia (CFU=colony forming units) before and after heat treatment at 85° C., for 1 hr. The results in Table 1 demonstrate that for each of the infectious fungi tested, the heat treatment reduced the culturable cell population 100 to 1000-fold, except for the R. stolonifer suspension, which produced no culturable cells on PDA.

TABLE 2

| Comparison | A. terreus | A. fumigatus | A. flavus | M. racemosus | R. stolonifer | P. variotii |
|---|---|---|---|---|---|---|
| A: Dead PMA - | | | | | | |
| Live PBS | | | | | | |
| Rep 1 | 9.19 | 6.82 | 8.4 | 5.59 | 8.2 | 7.17 |
| Rep 2 | 8.28 | 6.5 | 7.48 | 6.66 | 10.1 | 4.76 |
| Rep 3 | 7.74 | 5.68 | 7.77 | 8.23 | 9.18 | 6.35 |
| Mean | 8.4 | 6.33 | 7.88 | 6.83 | 9.16 | 6.09 |
| STD | 0.73 | 0.59 | 0.47 | 1.33 | 0.95 | 1.23 |
| upper 95% CI | 9.83 | 7.49 | 8.80 | 9.44 | 11.02 | 8.50 |
| lower 95% CI | 6.97 | 5.17 | 6.96 | 4.22 | 7.30 | 3.68 |
| B: Dead PBS - | | | | | | |
| Live PBS | | | | | | |
| Rep 1 | 0.75 | 0.62 | 1.21 | −0.87 | −0.26 | 0.9 |
| Rep 2 | 1.45 | 0.63 | 0.05 | −0.04 | −0.07 | 0.41 |
| Rep 3 | 1.2 | −0.66 | 0.02 | 0.25 | −0.97 | 0.06 |
| Mean | 1.2 | 0.2 | 0.43 | −0.22 | −0.43 | 0.46 |
| STD | 1.28 | 0.74 | 0.68 | 0.58 | 0.47 | 0.42 |
| upper 95% CI | 3.71 | 1.65 | 1.76 | 0.92 | 0.49 | 1.28 |
| lower 95% CI | −1.31 | −1.25 | −0.90 | −1.36 | −1.35 | −0.36 |
| C: Live PMA - | | | | | | |
| Live PBS | | | | | | |
| Rep 1 | 1.03 | 3.02 | 2.47 | 0.1 | 1.05 | 0.63 |
| Rep 2 | 1.81 | 0.59 | 1.92 | 1.53 | 0.14 | 0.2 |
| Rep 3 | 3.77 | 1.15 | 1.66 | 2.18 | −0.72 | −0.53 |
| Mean | 2.2 | 1.59 | 2.02 | 1.27 | 0.16 | 0.1 |
| STD | 1.41 | 1.27 | 0.41 | 1.06 | 0.89 | 0.59 |
| upper 95% CI | 4.96 | 4.08 | 2.82 | 3.35 | 1.90 | 1.26 |
| lower 95% CI | −0.56 | −0.90 | 1.22 | −0.81 | −1.58 | −1.06 |

Table 2 show Mean cycle threshold (CT) differences and standard deviation (STD) for the live and dead conidial suspensions, on filters exposed to either PMA or PBS. A 95% confidence interval (CI) is shown for each comparison. Table 2 shows the results of the application of the viability test to simulated air or water samples for each of the individual species. In QPCR, a 10-fold difference in concentration of organisms is equivalent to approximately 3 cycle threshold (CT) values (Haugland et al., 2004). The change measured in the viable population (“Dead-PMA” minus “Live-PBS”) is approximately 100 to 1000-fold, or approximately 6 to 9 CTs, as estimated by the PMA test (Table 2, Treatment A). These results are concordant with quantities estimated by culturing these same conidial suspensions (Table 1).

In order to demonstrate total recovery of conidia and DNA in the test, comparisons of “Dead-PBS” and “Live-PBS” treatments were evaluated (Table 2, Treatment B). The difference in CT was small (range 1.2 to −0.43), indicating good recovery of all of the cells/DNA. Finally, the comparison of the “Live-PMA” (i.e. not heat treated) minus “Live-PBS” indicates that a small part of the initial population of cells (about 10% or less, depending on fungal species) were dead before heat treatment.

TABLE 3

| Comparison | A. terreus | A. fumigatus | A. flavus | M. racemosus | R. stolonifer | P. variotii |
|---|---|---|---|---|---|---|
| A: Dead PMA - | | | | | | |
| Live PBS | | | | | | |
| Rep 1 | 8.37 | 7.71 | 7.71 | 7.41 | 11.5 | 6.44 |
| Rep 2 | 8.96 | 6.25 | 5.97 | 8.05 | 10.73 | 8.07 |
| Rep 3 | 7.54 | 8.18 | 7.78 | 6.79 | 7.97 | 5.26 |
| Mean | 8.29 | 7.38 | 6.88 | 7.42 | 10.07 | 6.59 |
| STD | 0.71 | 1.01 | 1.28 | 0.89 | 1.86 | 0.83 |
| upper 95% CI | 9.68 | 9.36 | 9.39 | 9.16 | 13.72 | 8.22 |
| lower 95% CI | 6.90 | 5.40 | 4.37 | 5.68 | 6.42 | 4.96 |
| B: Dead PBS - | | | | | | |
| Live PBS | | | | | | |
| Rep 1 | 1.15 | −0.24 | 0 | 1.65 | 0.77 | 1.24 |
| Rep 2 | 2.16 | 1.54 | 1.66 | 4.69 | 1.7 | 2.62 |
| Rep 3 | 0.16 | 0.6 | 0 | 1.67 | 0.06 | 0.44 |
| Mean | 1.16 | 0.63 | 0.55 | 2.67 | 0.84 | 1.43 |
| STD | 1 | 0.73 | 0.78 | 1.75 | 0.82 | 0.57 |
| upper 95% CI | 3.12 | 2.06 | 2.08 | 6.10 | 2.45 | 2.55 |
| lower 95% CI | −0.80 | −0.80 | −0.98 | −0.76 | −0.77 | 0.31 |
| C: Live PMA- | | | | | | |
| Live PBS | | | | | | |
| Rep 1 | 4.23 | 1.1 | 2.08 | 0.89 | 0.45 | 0.7 |
| Rep 2 | 5.51 | 1.99 | 3.6 | 2.71 | 1.32 | 2.18 |
| Rep 3 | 4.39 | 2.2 | 1.67 | 0.27 | 0.76 | 1.2 |
| Mean | 4.71 | 1.76 | 2.64 | 1.29 | 0.84 | 1.36 |
| STD | 0.11 | 0.58 | 1.36 | 1.27 | 0.22 | 0.35 |
| upper 95% CI | 4.93 | 2.90 | 5.31 | 3.78 | 1.27 | 2.05 |
| lower 95% CI | 4.49 | 0.62 | −0.03 | −1.20 | 0.41 | 0.67 |

Table 3 shows the mean cycle threshold (CT) differences and standard deviation (STD) for the live and dead mixed conidial suspensions, on filters exposed to either PMA or PBS. A 95% confidence interval (CI) is shown for each comparison. The results in Table 3 show that, even when the conidial suspensions were mixed together before treatment, the difference in CTs (Table 3, Treatment A) were approximately the same, as seen with the individual species (Table 2). “Dead-PBS” minus “Live-PBS” again showed good recovery of the cells/DNA (Table 3, Treatment B). Comparison of “Live-PMA” minus “Live-PBS” CT results showed that some of the initial populations were already dead, even before heat treatment (Table 3, Treatment C). These results are consistent with the results of the individual species.

Treatment of simulated environmental samples with PMA was very effective at estimating populations of live and dead infectious fungal conidia. When PMA discrimination of live and dead cells is combined with QPCR analysis of environmental samples, the process from sample to result can be obtained in about 2 hrs. This compares with days to weeks to obtain results from culturing. Time-to-results may be very important in monitoring air and water for infectious fungi, especially in the environments of the immuno-compromised, since fungal infections or mycoses are on the rise.

Although a few exemplary embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A microorganism discriminator, comprising:
a housing to incubate a sample in low-light conditions;
an injector disposed in the housing, to deliver a viability discriminating dye to the sample;
an illuminator to radiate the sample with blue light; and
a base to transport the sample between the housing and the illuminator.

2. The discriminator of claim 1, wherein the illuminator comprises:
a light source to emit the blue light; and
a lens to focus the blue light onto the sample.

3. The discriminator of claim 2, wherein the light source comprises at least one blue light emitting diodes.

4. The discriminator of claim 2, wherein the illuminator further comprises a driver to oscillate the light source and the lens.

5. The discriminator of claim 2, wherein the lens is a Fresnel lens.

6. The discriminator of claim 1, wherein the viability discriminating dye comprises Propidium monoazide (PMA).

7. The discriminator of claim 1, further comprising a frame connected to the base, to move the housing with respect to the base.

8. The discriminator of claim 7, wherein the frame comprises an elevator to move the housing toward the base and away from the base.

9. The discriminator of claim 1, further comprising a conveyor disposed on the base, to move the sample to the housing and to the illuminator.

10. The discriminator of claim 1, wherein the blue light has a wavelength ranging from about 445 nm to about 485 nm.

11. The discriminator of claim 1, wherein the injector comprises an array of pipettors.

12. The discriminator of claim 1, further comprising:
a slide having a well including a liquid buffer, upon which a filter including the sample is disposed; and
a sample plate to hold the slide.

13. The discriminator of claim 12, wherein a plurality of the slides are disposed on the sample plate.

14. A method of discriminating viable microorganisms in a sample, the method comprising:
applying a viability discriminating dye to a filter comprising a sample, in a low-light environment;
incubating the sample and dye in the low-light environment;
illuminating the incubated sample and dye with monochromatic blue light; and
performing quantitative polymerase chain reaction (QPCR) on the previously illuminated sample.

15. The method of claim 14, further comprising disposing the filter over a well of a slide, the well containing a buffer.

16. The method of claim 14, wherein the blue light has a wavelength ranging from about 445 nm to about 485 nm.

17. The method of claim 14, wherein the blue light has a wavelength of about 470 nm.

18. The method of claim 14, wherein the viability discriminating dye comprises Propidium monoazide (PMA).

19. The method of claim 14, wherein the illuminating comprises using at least one blue light emitting diode to produce the blue light, and collecting and focusing the blue light with a Fresnel lens.

20. The method of claim 14, wherein the illuminating comprises oscillating a light source above the filter.

21. A microorganism discriminator, comprising:
a housing to incubate a sample in low-light conditions;
an injector disposed in the housing, to deliver a viability discriminating dye to the sample; and
an illuminator to radiate the sample with monochromatic blue light.

22. The microorganism discriminator of claim 21, wherein the illuminator comprises:
a light source to emit the monochromatic blue light to the sample plate; and
a Fresnel lens to focus the blue light onto the sample.

23. The microorganism discriminator of claim 21, further comprising:
a base connected to the housing and the illuminator, to transport the sample between the housing and to the illuminator.

24. The microorganism discriminator of claim 23, further comprising:
a frame connected to the housing and the base; and
an elevator to disposed on the frame, to move the housing with respect to the base.
